(19) United States
(12) Patent Application Publication
(10) Pub. No.: US 2025/0082558 A1
(43) Pub. Date: Mar. 13, 2025

(54) COSMETIC PRODUCT OF PACKAGING AND A PREPARATION CONTAINING 1,2-PROPANE DIOL ESTER

(71) Applicant: BEIERSDORF AG, Hamburg (DE)

(72) Inventors: Claudia MUELLER, Tangstedt (DE); Heike VOLBRICH, Hamburg (DE); Nicole KLESY, Hamburg (DE); Alexis GOULET-HANSSENS, Hamburg (DE)

(21) Appl. No.: 18/580,223

(22) PCT Filed: Jun. 22, 2022
(86) PCT No.: PCT/EP2022/066995

§ 371 Date: Jan. 18, 2024

(30) Foreign Application Priority Data
Jul. 20, 2021 (DE) 10 2021 207 728.5

(51) Int. Cl. A61K 8/37; A61K 8/06; A61Q 19/00

(52) U.S. Cl. CPC A61K 8/375; A61K 8/062; A61Q 19/00; A61K 2800/34; A61K 2800/87

(57) ABSTRACT

Cosmetic product of a) a packaging of high-density polyethylene (HDPE), low-density polyethylene (LDPE), polypropylene (PP) or polyethylene terephthalate (PET), as well as b) a cosmetic preparation containing propane-1,2-diol ester.

The present invention relates to a cosmetic product of a cosmetic preparation comprising propane-1,2-diol esters and a packaging of HDPE, LDPE, PP or PET.

The desire to look beautiful and attractive is naturally rooted in humans. Although ideals of beauty have changed over time, the pursuit of a flawless appearance has always been aimed for by humans. An essential part of a beautiful and attractive appearance is the condition and complexion of the skin.

In order for the skin to be able to perform the full range of its biological functions, it requires regular cleansing and care. Cleansing of the skin serves to remove dirt, sweat and residual dead skin particles, which form an ideal nutrient source for all kinds of germs and parasites. Skincare products mostly serve for moisturizing and refatting the skin. Active ingredients are commonly added thereto, which are intended to regenerate the skin and for example to prevent and reduce the premature aging thereof (for example the appearance of fine lines and wrinkles).

Skincare products are generally stored in packagings in the form of bottles, tubes or jars. These predominantly consist of glass (glass jars) or plastics such as high-density polyethylene (HDPE), low-density polyethylene (LDPE), polypropylene (PP) or polyethylene terephthalate (PET).

A disadvantage of such inherently very inexpensive and extremely hygienic packagings is the fact that these packagings are not particularly sustainable/environmentally friendly, since they are produced from petroleum products and/or natural gas and their thermal utilization (incineration in a waste incineration plant) ultimately releases carbon dioxide from fossil sources.

In order to conserve natural resources, attempts are therefore being made to produce these packagings as thin-walled as possible and to recycle them after the preparation has been used.

However, these thin-walled packagings have the disadvantage that, if the cosmetic preparation with which they are filled comprises one or more oil components, said packagings are not particularly storage-stable, but rather swell on contact with the oil, since the oil is “sucked up” and absorbed by the plastic. Moreover, oil may separate out of the products. Furthermore, the oil in the plastic leads to lettering printed on the packaging being able to smudge and rub off. In addition, the oil absorbed by the packaging makes it difficult to recycle the plastic because the oil has to be laboriously separated from the polymer before a new packaging can be produced again from the polymer plastic. For these reasons, either only oil-free preparations (e.g. toothpaste) can be used to fill conventional, thin-walled packagings, or the packaging must be elaborately coated or sealed on the inner side facing the cosmetic product. Known from the prior art, for example, are protective barriers made of ethylene vinyl alcohol, which are relatively expensive and also less easy to recycle than pure polyolefin packaging.

The problem of the absorption of oils from the preparation by the packaging is further exacerbated by the general trend of certain oil components from industrial production being banned from cosmetic preparations, for example due to their poor degradability or high emission values during production. Dimethicone in particular was/is particularly popular with product developers per se due to its low tendency to migrate into plastic packagings. On the other hand, there are various reasons that require cosmetic preparations to be formulated without dimethicone.

It was therefore the object of the present invention to reduce and ideally to eliminate the disadvantages of the prior art and to develop a cosmetic product consisting of an oil-containing cosmetic preparation and a plastic packaging, the packaging of which is easier to recycle and is storage-stable, i.e., in which the oil absorption of the plastic packaging is significantly reduced, without it being necessary to apply a protective layer (for example made of ethylene vinyl alcohol) to the plastic.

The object is surprisingly achieved by a cosmetic product of
a) a packaging of high-density polyethylene (HDPE), low-density polyethylene (LDPE), polypropylene (PP) or polyethylene terephthalate (PET), and
b) a cosmetic preparation containing propane-1,2-diol ester.

According to the invention, it is possible here to use either one or more of these propane-1,2-diol esters.

Although those skilled in the art are familiar with cosmetic products of packagings with HDPE, PP or PET which contain cosmetic preparations with ester oils, such as C12-15 alkyl benzoate, or ether oils, such as dicaprylyl ether, these oil components of the prior art tend to migrate relatively greatly into these packaging materials. Therefore, such products usually have a protective layer made of, for example, ethylene vinyl alcohol on the inner side, which is usually not declared or does not have to be declared on the packaging.

It is preferred according to the invention if the packaging a) is formed from high-density polyethylene (HDPE).

Furthermore, it is advantageous according to the invention if the packaging a) has a wall thickness of 1 to 8 mm.

Moreover, the embodiments of the present invention that are advantageous according to the invention are characterized in that the packaging does not have any protective layer (for example of ethylene vinyl alcohol) on the inner side facing the preparation.

It is preferred according to the invention if propylene glycol dicaprate (INCI: Propylene Glycol Dicaprate), propylene glycol dicaprylate/dicaprate (INCI: Propylene Glycol Dicaprylate/Dicaprate), propylene glycol diheptanoate (INCI: Propylene Glycol Diheptanoate), propylene glycol dibenzoate (INCI: Propylene Glycol Dibenzoate) and/or dipropylene glycol dibenzoate (INCI: Propylene Glycol Dibenzoate) is/are used as propane-1,2-diol esters.

Embodiments of the present invention that are advantageous according to the invention are also obtained by the preparation being free of dimethicone (INCI: Dimethicone).

Moreover, it is advantageous according to the invention if the preparation is free of silicone oil, mineral oil, paraffin wax, microcrystalline wax, shellac wax and polyethylene waxes.

It is also advantageous according to the invention if the preparation is free of polyacrylates, crosslinked acrylate/C10-C30 alkyl acrylate polymers and vinylpyrrolidone/hexadecene copolymers, and free of 3-(4-methylbenzylidene) camphor, 2-hydroxy-4-methoxybenzophenone (INCI: Oxybenzone), 2-ethylhexyl 4-methoxycinnamate (INCI: Octyl Methoxycinnamate), ethylhexyl 2-cyano-3,3-diphenylacrylate (INCI: Octocrylene), parabens (particularly methyl, propyl and butyl paraben), methylisothiazolinone, chloromethylisothiazolinone and DMDM hydantoin, polyethylene glycol ethers or polyethylene glycol esters.

It is also advantageous in the sense of the present invention if the cosmetic preparation is in the form of an emulsion. It is preferred according to the invention here if the cosmetic preparation is in the form of an oil-in-water emulsion (O/W emulsion).

It is advantageous according to the invention if the cosmetic preparation contains propane-1,2-diol esters at a concentration of 1.0% to 15% by weight, based on the total weight of the preparation. Preferred according to the invention here is a use concentration of 1% to 5% by weight, based on the total weight of the preparation. These use concentrations relate to the total amount of propane-1,2-diol esters used. If only one propane-1,2-diol ester (for example propylene glycol dicaprate (INCI: Propylene Glycol Dicaprate), propylene glycol dicaprylate/dicaprate (INCI: Propylene Glycol Dicaprylate/Dicaprate), propylene glycol diheptanoate (INCI: Propylene Glycol Diheptanoate), propylene glycol dibenzoate (INCI: Propylene Glycol Dibenzoate), dipropylene glycol dibenzoate (INCI: Propylene Glycol Dibenzoate)) is used, said concentration is the advantageous or preferred use concentration for this one compound.

It is also advantageous according to the invention if the total amount of propane-1,2-diol esters in the oil phase of the preparation is at least 3% by weight, based on the total weight of the oil phase.

Embodiments that are advantageous according to the invention are also characterized in that the preparation contains one or more oils selected from the group of the compounds Octyldodecanol, Caprylic/Capric Triglyceride (INCI: Caprylic/Capric Triglyceride), cocoglycerides (INCI: Cocoglycerides), tridecyl stearate (INCI: Tridecyl Stearate), tridecyl trimellitate (INCI: Tridecyl Trimellitate).

Furthermore, it is advantageous according to the invention if the preparation contains one or more oils selected from the group of compounds containing polycitronellol (INCI: Polycitronellol), polycitronellol acetate (INCI: Polycitronellol Acetate), triolein, ethylhexyl acetoxystearate (INCI: Ethylhexyl Acetoxystearate) and/or acetyl ethylhexyl polyhydroxystearate (INCI: Acetyl Ethylhexyl Polyhydroxystearate).

It is also advantageous according to the invention if the preparation contains one or more oils selected from the group of the compounds almond oil (INCI: Prunus amygdalus dulcis Oil) and/or sunflower oil (INCI: Helianthus annuus Seed Oil), hydrogenated castor oil (INCI: Hydrogenated Castor Oil).

Moreover, it is advantageous according to the invention if the preparation contains tocopherol and/or tocopherol acetate.

Last but not least, it is advantageous according to the invention if the preparation contains ethanol and/or ethylhexylglycerin.

The preparation according to the invention may otherwise contain the ingredients customary for cosmetic products and be composed accordingly.

### COMPARATIVE EXPERIMENTS

The penetration of the cosmetic oils that follow into the packaging material HDPE was investigated. The HDPE used in this case was the product “Hostalen ACP 5831D” from Lyondellbasell.

### Test Procedure

1. Use three die-cuts from the packaging material having a diameter of 5 mm and a height of 1 mm for each raw material/each cosmetic preparation.
2. Determine the initial weight of each die-cut on an analytical balance. (Initial weight=m0)
3. Place the die-cuts in a sealable vessel and add the raw material/the cosmetic preparation. The die-cuts must be completely enveloped by the raw material/the cosmetic preparation.
4. Store the closed vessel in an incubator at 40±1° C.
5. Mechanically clean the die-cuts with cellulose after 28 days.
5. Reweigh the die-cuts after 28 days.

| Weight of the sample (die-cut) | Determination |
|---|---|
| m0 | Initial weight |
| m1 | 28 days |

### Notes

Do not touch the die-cuts with your fingers; always use tweezers to avoid contamination.

### Evaluation

Raw materials or constituents of the cosmetic preparation have migrated into the packaging material if the weight of the die-cut m1 is greater than the weight of m0.

$$Weight increase ( \% ) = ( m 1 - m 0 ) / m 0$$

The formula (m1−m0)/m0 is used to determine the weight increase (%) of the raw material or of the cosmetic preparation which has migrated into the packaging material.

Mean value [%] and standard deviation [%] of the triple determination are additionally calculated.

### Results

| | Weight increase in | |
|---|---|---|
| | % after 28 d | |
| | Mean | Standard |
| INCI | value [%] | deviation [%] |
| Propylene glycol dicaprate (INCI: | 3.34 | 0.01 |
| Propylene Glycol Dicaprate) | | |
| Propylene glycol dicaprylate/dicaprate | 3.30 | 0.01 |
| (INCI: Propylene Glycol Dicaprylate/ | | |
| Dicaprate) | | |
| Propylene glycol diheptanoate (INCI: | 3.18 | 0.02 |
| Propylene Glycol Diheptanoate) | | |
| Propylene glycol dibenzoate (INCI: | 1.16 | 0.04 |
| Propylene Glycol Dibenzoate) | | |
| Dipropylene glycol dibenzoate (INCI: | 0.90 | 0.06 |
| Propylene Glycol Dibenzoate) | | |
| Paraffinum Liquidum | 5.24 | 0.05 |
| C12-15 Alkyl benzoate | 4.85 | 0.04 |
| Dicaprylyl ether | 5.52 | 0.04 |

### EXAMPLES

The examples which follow are intended to illustrate the present invention without limiting it. Unless otherwise indicated, all amounts, proportions and percentages are based on the weight and the total amount or on the total weight of the preparations.

| | Sample | Sample | Sample | Sample | Sample | Sample | Sample | Sample |
|---|---|---|---|---|---|---|---|---|
| INCI | 1 | 2 | 3 | 4 | 5 | 6 | 7 | 8 |
| Glycyrrhetinic Acid | 0.1 | 0.1 | | | | | | |
| Tocopheryl Acetate | 0.1 | 0.1 | 0.1 | 0.1 | 0.1 | 0.1 | 0.1 | 0.1 |
| Glycyrrhiza Inflata Root | | | 0.05 | 0.05 | | | | |
| Extract | | | | | | | | |
| Ethylhexylglycerin | | | 0.3 | 0.3 | | | | |
| Hydroxyacetophenone | | | | | 0.4 | 0.4 | | |
| Dibutyl Adipate | 4.0 | 4.0 | | | | | 3.0 | 3.0 |
| Propylene Glycol | | | 2.0 | | 2.0 | 3.0 | | 1.5 |
| Dicaprate | | | | | | | | |
| Propylene Glycol | | | 3.0 | 5.0 | | 4.0 | 4.0 | |
| Diheptanoate | | | | | | | | |
| Propylene Glycol | 9.5 | 4.5 | 2.0 | | 2.0 | | | |
| Dibenzoate | | | | | | | | |
| Dipropylene Glycol | | 5.0 | | 1.5 | 0.5 | | | 1.5 |
| Dibenzoate | | | | | | | | |
| Caprylic/Capric | 15.0 | 15.0 | 2.0 | 5.0 | | | 1.5 | |
| Triglyceride | | | | | | | | |
| Butylene Glycol | 5.2 | 5.2 | 4.0 | 4.0 | 2.0 | | 2.0 | 2.0 |
| Dicaprylate/Dicaprate | | | | | | | | |
| Dicaprylyl Carbonate + | 2.0 | 2.0 | | | 0.0 | 2.0 | 0.0 | 0.0 |
| Tocopherol | | | | | | | | |
| Isopropyl Palmitate | | | | | 5.5 | 2.0 | 3.0 | 4.0 |
| Hydrogenated Rapeseed | | | | | 2.0 | 2.0 | 0.5 | 0.5 |
| Oil | | | | | | | | |
| Cetyl Palmitate | | | | | | | 0.4 | 0.4 |
| Copernicia Cerifera | | | | | | | 0.5 | 0.5 |
| Cera | | | | | | | | |
| Glyceryl Stearate | | | 0.8 | 0.8 | | | 1.0 | 1.0 |
| Sodium Stearoyl | | | 0.4 | 0.4 | 0.3 | 0.3 | 0.4 | 0.4 |
| Glutamate + Sodium | | | | | | | | |
| Chloride | | | | | | | | |
| Distarch Phosphate | | | 2.0 | 2.0 | | | | |
| Silica Dimethyl Silylate | | | | | 1.0 | 1.0 | | |
| Glycerin | 2.5 | 2.5 | 6.0 | 6.0 | 8.0 | 8.0 | 5.0 | 5.0 |
| Caprylyl Glycol + Aqua | | | | | | | 0.2 | 0.2 |
| Phenoxyethanol + Aqua | | | 0.7 | 0.7 | 0.5 | 0.5 | 0.4 | 0.4 |
| Aqua + Trisodium | | | 1.0 | 1.0 | 1.0 | 1.0 | 1.0 | 1.0 |
| EDTA | | | | | | | | |
| Alcohol Denat. + Aqua | 49.3 | 49.3 | 4.0 | 4.0 | 5.0 | 5.0 | 7.0 | 7.0 |
| Cetearyl Alcohol | | | 2.0 | 2.0 | | | | |
| Xanthan Gum | | | 0.1 | 0.1 | 0.4 | 0.4 | 0.2 | 0.2 |
| Behenyl Alcohol | | | | | 0.9 | 0.9 | | |
| Stearyl Alcohol | | | | | 0.9 | 0.9 | | |
| Cellulose Gum | | | | | 0.5 | 0.5 | | |
| Microcrystalline | | | | | | | 1.0 | 1.0 |
| Cellulose + Cellulose | | | | | | | | |
| Gum | | | | | | | | |
| Diethylamino | 2.0 | 3.0 | 1.0 | 1.0 | 2.0 | 1.0 | 3.0 | 2.0 |
| Hydroxybenzoyl Hexyl | | | | | | | | |
| Benzoate | | | | | | | | |
| Bis- | 3.0 | 4.0 | 3.5 | 3.5 | 3.0 | 4.0 | | 3.0 |
| Ethylhexyloxyphenol | | | | | | | | |
| Methoxyphenyl Triazine | | | | | | | | |
| Ethylhexyl Triazone | 2.5 | 1.0 | 3.5 | 3.5 | 1.0 | 2.0 | 3.0 | 3.0 |
| Butyl | 3.3 | 3.3 | 4.8 | 4.8 | 4.8 | 4.8 | 3.0 | 3.0 |
| Methoxydibenzoyl- | | | | | | | | |
| methane | | | | | | | | |
| Phenylbenzimidazole | 0.0 | 0.0 | 2.0 | 2.0 | 1.0 | 0.5 | 0.5 | 0.5 |
| Sulfonic Acid | | | | | | | | |
| Titanium Dioxide | | | | | 3.0 | 3.0 | | |
| (nano) + Silica + | | | | | | | | |
| Dimethicone | | | | | | | | |
| add Water, Perfume and | 100.0 | 100.0 | 100.0 | 100.0 | 100.0 | 100.0 | 100.0 | 100.0 |
| NaOH | | | | | | | | |

These preparations were filled into and stored in a bottle-shaped storage vessel made of HDPE (Hostalen ACP 5831 D, from Lyondellbasell).

1.-17. (canceled)

18. A cosmetic product, wherein the product comprises
(a) a packaging made of high-density polyethylene (HDPE), low-density polyethylene (LDPE), polypropylene (PP) or polyethylene terephthalate (PET), and
(b) a cosmetic preparation comprising one or more propane-1,2-diol esters.

19. The cosmetic product of claim 18, wherein (a) is made of high-density polyethylene (HDPE).

20. The cosmetic product of claim 18, wherein (a) has a wall thickness of from 1 to 8 mm.

21. The cosmetic product of claim 18, wherein (a) does not have any protective layer on an inner side thereof that is in contact with (b).

22. The cosmetic product of claim 18, wherein (b) comprises one or more of propylene glycol dicaprate, propylene glycol dicaprylate/dicaprate, propylene glycol diheptanoate, propylene glycol dibenzoate or dipropylene glycol dibenzoate.

23. The cosmetic product of claim 18, wherein (b) is free of dimethicone.

24. The cosmetic product of claim 18, wherein (b) is free of silicone oil, mineral oil, paraffin wax, microcrystalline wax, shellac wax and polyethylene waxes.

25. The cosmetic product of claim 18, wherein (b) is free of polyacrylates, crosslinked acrylate/C10-C30 alkyl acrylate polymers and vinylpyrrolidone/hexadecene copolymers, 3-(4-methylbenzylidene) camphor, 2-hydroxy-4-methoxybenzophenone, 2-ethylhexyl 4-methoxycinnamate, ethylhexyl 2-cyano-3,3-diphenylacrylate, parabens, methylisothiazolinone, chloromethylisothiazolinone, DMDM hydantoin, polyethylene glycol ethers and polyethylene glycol esters.

26. The cosmetic product of claim 25, wherein (b) is free of dimethicone, silicone oil, mineral oil, paraffin wax, microcrystalline wax, shellac wax and polyethylene waxes.

27. The cosmetic product of claim 18, wherein (b) is present in the form of an emulsion.

28. The cosmetic product of claim 27, wherein (b) is present in the form of an oil-in-water emulsion (O/W emulsion).

29. The cosmetic product of claim 18, wherein (b) comprises the one or more propane-1,2-diol esters at a concentration of from 1% to 15% by weight, based on a total weight of the preparation.

30. The cosmetic product of claim 22, wherein (b) comprises the one or more propane-1,2-diol esters at a concentration of from 1% to 15% by weight, based on a total weight of the preparation.

31. The cosmetic product of claim 18, wherein a total concentration of the one or more propane-1,2-diol esters in an oil phase of the preparation is at least 3% by weight, based on a total weight of the oil phase.

32. The cosmetic product of claim 29, wherein a total concentration of the one or more propane-1,2-diol esters in an oil phase of the preparation is at least 3% by weight, based on a total weight of the oil phase.

33. The cosmetic product of claim 18, wherein (b) further comprises one or more of octyldodecanol, caprylic/capric triglyceride, cocoglycerides, tridecyl stearate, tridecyl trimellitate.

34. The cosmetic product of claim 18, wherein (b) further comprises one or ore oils selected from polycitronellol, polycitronellol acetate, triolein, ethylhexyl acetoxystearate, acetyl ethylhexyl polyhydroxystearate.

35. The cosmetic product of claim 18, wherein (b) further comprises tocopherol and/or tocopherol acetate.

36. The cosmetic product of claim 18, wherein (b) further comprises one or more oils selected from almond oil, sunflower oil, hydrogenated castor oil.

37. The cosmetic product of claim 18, wherein (b) further comprises ethanol and/or ethylhexylglycerin.
